(19) United States
(12) Patent Application Publication
(10) Pub. No.: US 2009/0259283 A1
(43) Pub. Date: Oct. 15, 2009

(54) SHEATHED LEAD FOR PACING OR DEFIBRILLATION

(76) Inventors: Michael S. Brandt, Shoreview, MN (US); Bruce A. Tockman, Scandia, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/396,695

(22) Filed: Mar. 3, 2009

Related U.S. Application Data
(60) Provisional application No. 61/043,482, filed on Apr. 9, 2008.

(51) Int. Cl. A61N 1/05

(52) U.S. Cl. 607/127

(57) ABSTRACT

A cardiac lead assembly includes a sheath disposed over a majority of the outer surface of a cardiac lead body including a fixation helix located at its distal end. The sheath protects the fixation member during delivery of the lead assembly to a target location within a patient's heart. The sheath can include one or more means adapted for splitting the sheath such that the sheath can be removed from about the lead body once the lead has been implanted.

### CROSS-REFERENCE TO RELATED APPLICATION

This application claims priority under 35 U.S.C. §119 to U.S. Provisional Application No. 61/043,482, filed on Apr. 9, 2008, entitled “Sheathed Lead For Pacing Or Defibrillation,” which is incorporated herein by reference in its entirety.

### TECHNICAL FIELD

The present invention relates to implantable cardiac leads. More particularly, the present invention relates to a system and a method for delivering and implanting a screw-in cardiac lead within a patient's heart.

### BACKGROUND

Implantable medical devices for treating irregular contractions of the heart with electrical stimuli are well known. Exemplary examples of such devices, which include defibrillators and pacemakers, generally include a medical electrical lead for delivering electrical stimulation therapy to the heart connected to a pulse generator. Such leads are introduced into the patient's vasculature at a venous access site and travel through veins to the sites where the leads' electrodes will be implanted or otherwise contact target coronary tissue. Important characteristics of the medical electrical leads include biocompatibility, durability, reduced diameter, and handling characteristics.

### SUMMARY

According to various embodiments, the present invention is a cardiac rhythm management system including a pulse generator and a cardiac lead assembly adapted to be connected to a pulse generator. According to various embodiments of the present invention, the cardiac lead assembly includes a cardiac lead having an elongated lead body including an outer surface and a proximal end and a distal end, at least one electrode located on the lead body, at least one conductor operatively connected to the at least one electrode, and a fixation helix located at the distal end of the lead body, the fixation helix including a distal end. Additionally, the cardiac lead assembly includes a retractable sheath disposed over at least a portion of the outer surface of the lead body and the fixation helix located at the distal end of the lead body. According to some embodiments, the retractable sheath is a splittable sheath adapted to be split for removal of the sheath from about the outer surface of the lead body. According to other embodiments, the sheath can be retracted and then remain in a retracted position over the outer surface of the lead body. Additionally, in some embodiments, a distal end of the sheath can be at least flush with a distal end of the fixation helix prior to retraction of the sheath.

According to various embodiments, the sheath includes one or more means for splitting the sheath extending from a proximal end to a distal end of the sheath. Additionally, according to further embodiments, the sheath can include wings or tabs located at the proximal end of the sheath to assist a physician in splitting the sheath. In some embodiments, the sheath may be removed after the lead has been implanted at the target location within a patient's heart. According to other embodiments, the sheath can remain disposed over the lead body for chronic implantation of the lead. According to this embodiment, the sheath may be removed at a later time.

According to other embodiments, the present invention is a method of delivering a cardiac lead to a target location within a patient's heart. According to various embodiments, the method includes introducing a cardiac lead assembly into the patient's vasculature. The cardiac lead assembly includes a lead body having a distal end and a fixation helix located at the distal end of the lead body and including a helix distal end. A retractable sheath is disposed over the lead body and includes a sheath proximal end and a sheath distal end, the sheath distal end positioned at least flush with the helix distal end. Additionally, the method includes; retracting the sheath to expose the fixation helix; evaluating pacing thresholds at the target location; and screwing the fixation helix into cardiac tissue at the target implantation site. According to further embodiments, the method includes removing the sheath from about the lead body. According to yet other embodiments, the method includes securing the retracted sheath in a retracted position. According to further embodiments, the cardiac lead assembly can be inserted into a patient's vasculature using an introducer.

According to other embodiments, the present invention is a cardiac lead assembly including a cardiac lead and a sheath. According to various embodiments, the cardiac lead includes: an elongate lead body having an outer surface and a proximal end and a distal end; at least one electrode located on the lead body; at least one conductor operatively connected to the at least one electrode; and a fixation helix located at the distal end of the body, the fixation helix including a distal end. According to various embodiments the sheath is disposed over at least a portion of the outer surface of the lead body and the fixation helix. The sheath includes a proximal end and a distal end and splitting means for longitudinally splitting the sheath between the proximal and distal ends. According to some embodiments, the means for splitting the sheath can include any one of a line of weakness, a line of perforations, a peel-away strip, or a line of score marks. According to yet further embodiments of the present invention, the sheath can include wings or tabs located at the proximal end of the sheath to assist a physician in splitting the sheath.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of a pulse generator coupled to a cardiac lead assembly deployed in a patient's heart according to various embodiments of the present invention.

FIGS. 2A and 2B are partial cross-sectional views of a cardiac lead assembly according to various embodiments of the present invention.

FIGS. 3A and 3B are perspective views of an outer sheath according to various embodiments of the present invention.

FIG. 4A and 4B are schematic views of a cardiac lead assembly according to other embodiments of the present invention.

FIG. 5 is a flow chart showing a method of implanting a cardiac lead assembly according to various embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives failing within the scope of the invention as defined by the appended claims.

### DETAILED DESCRIPTION

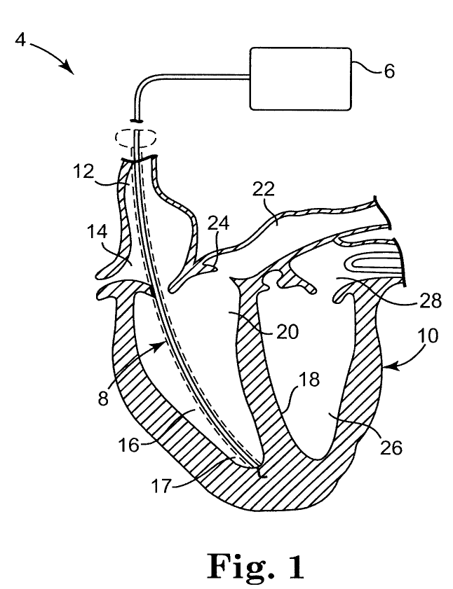

FIG. 1 shows a cardiac rhythm management system 4 according to various embodiments of the present invention. The cardiac rhythm management system 4 includes a pulse generator 6 coupled to a cardiac lead assembly 8 deployed in a patient's heart 10. As shown in FIG. 1, the heart 10 includes a superior vena cava 12, a right atrium 14, a right ventricle 16 having an apex 17, a ventricular septum 18, a ventricular outflow tract 20, which leads to a pulmonary artery 22 having a pulmonary valve 24, a left ventricle 26, and a left atrium 28. In one embodiment, the cardiac lead assembly 8 is positioned in the right ventricle 16 near the right ventricular apex 17. It is appreciated that the cardiac lead assembly 8, according to the various embodiments of the present invention, can be deployed to alternate locations within the heart.

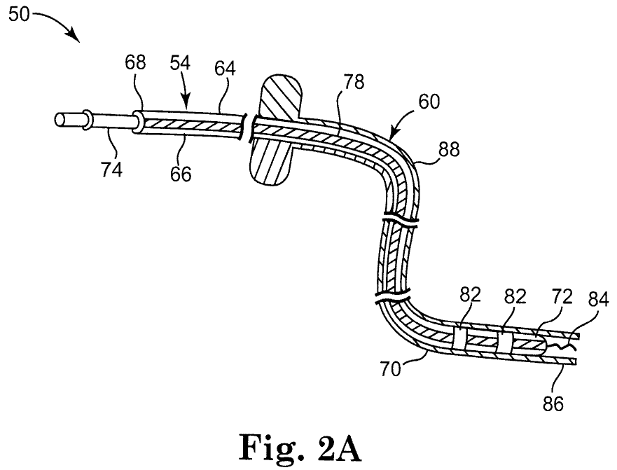

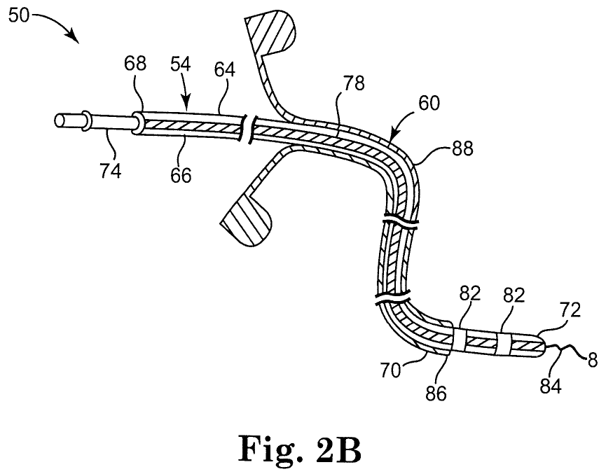

FIGS. 2A and 2B are partial cross-sectional views of a cardiac lead assembly 50 suitable for use in the CRM system 4 of FIG. 1, according to various embodiments of the present invention. As shown in FIGS. 2A and 2B, the cardiac lead assembly 50 includes a cardiac lead 54 and an outer sheath 60. The cardiac lead 54 includes an elongated, insulative lead body 64 extending from a proximal portion 66 including a proximal end 68 to a distal portion 70 including a distal end 72. The proximal end 68 is configured to be operatively connected to a pulse generator via a connector 74. At least one conductor 78 extends from the connector 74 at the proximal end 68 of the lead body 64 to one or more electrodes 82 at the distal end 72 of the lead body 64. The conductor 78 can be a coiled or cable conductor or wire. According to some embodiments where multiple conductors are employed, the lead can include a combination of coiled and cable conductors or wires. When a coiled conductor is employed, according to some embodiments, the conductor 78 can have either a co-radial or a co-axial configuration.

The lead body 64 is flexible, and has a substantially circular cross-section. According to various embodiments of the present invention, an outer diameter of the lead body 64 ranges from about 2 to about 9 French. The cardiac lead 54 may be unipolar, bipolar, or multi-polar depending upon the type of therapy to be delivered. In embodiments of the present invention employing multiple electrodes 82 and multiple conductors 78, each conductor 78 is adapted to be connected to an individual electrode 82 in a one-to-one manner allowing each electrode 82 to be individually addressable. Additionally, the lead body 64 can include one or more lumens. In some embodiments at least one lumen is adapted to receive the insertion of a conductor during construction of the medical electrical lead. In further embodiments, at least one lumen is adapted to receive a guiding element such as a guidewire or a stylet for delivery of the cardiac lead assembly 50 to a target location within a patient's heart. In various other embodiments, at least one of the conductors is a coil conductor defining a lumen adapted for receiving a guidewire and/or a stylet.

The electrodes 82 can have any electrode configuration suitable for use as a pacing and/or sensing electrode within a cardiac rhythm management system. According to one embodiment of the present invention, at least one electrode 82 is a ring or partial ring electrode. According to another embodiment, at least one electrode 82 is a shocking coil. According to yet another embodiment of the present invention, at least one electrode 82 includes an exposed electrode portion and an insulated electrode portion. In some embodiments, a combination of electrode configurations may be used. The electrodes 82 can be coated with or formed from platinum, stainless steel, MP35N, a platinum-iridium alloy, or another similar conductive material. In further embodiments, a steroid eluting collar may be located adjacent to at least one electrode 82.

According to various embodiments, the lead body 54 includes a fixation helix 84 for securing and stabilizing the lead body 54 including the one or more electrodes 82 at a target site within a patient's body. The fixation helix 84 can be electrically active or passive. In some embodiments, the fixation helix can be a screw-in fixation member 84 and is fixed relative to the distal end 72 of the lead body 54.

In the embodiment illustrated in FIGS. 2A and 2B, the outer sheath 60 is dimensioned such that it can extend over a majority of an outer surface 88 of the lead body 54. According to other embodiments, the outer sheath 60 is dimensioned such that it can extend over the entire outer surface 88 of the lead body 54. In some embodiments, the outer sheath 60 is dimensioned such that it can be disposed over about 50 to about 80 percent of the outer surface 88 of the lead body 54. According to various embodiments, the outer sheath 60 is dimensioned such that it can be disposed over the lead body 54 such that the proximal portion 66 of the lead body 54 is exposed, enabling a physician or other clinician to grasp the proximal portion 66 of the lead body 54 to manipulate the cardiac lead assembly 50 during implantation.

The outer sheath 60 operates in part to protect the fixation helix 84 during implantation. According to various embodiments, the sheath 60 is slideably disposed over and coupled to the lead body 54 such that the sheath 60 is moveable along a length of the lead body 54. As such, the outer sheath 60 is adapted to transition from at least an extended position in which the outer sheath 60 is disposed over the fixation helix 84, as shown in FIG. 2A, and a retracted position in which the outer sheath 60 has been retracted such that the fixation helix 84 and/or any electrodes 82 are fully exposed, as shown in FIG. 2B. In various embodiments of the present invention, the outer sheath 60 is dimensioned such that the distal end 86 of the outer sheath 60 can extend beyond the distal end 88 of the fixation helix 84 by a distance (e.g. ranging from about 0.1 to about 5.0 mm) sufficient to prevent the helix 84 from catching or contacting tissue during delivery. According to other embodiments, the sheath 60 can be positioned such that the distal end 86 of the sheath 60 is positioned flush with the distal end 88 of the fixation helix 84 to facilitate threshold measurements when the lead body 54 and sheath 60 are pressed against the myocardium. Once proper placement of the cardiac lead 50 has been determined, the fixation helix 84 can be secured at the implantation site, thereby anchoring and stabilizing the distal end portion of the lead 50 within the patient's heart.

According to further embodiments the outer sheath 60 may include one or more apertures adapted to be aligned with the one or more electrodes 82 located on the lead body 64. The apertures allow the exposed electrodes 82 to be utilized for evaluating pacing thresholds after retraction of the sheath to expose the fixation helix 84.

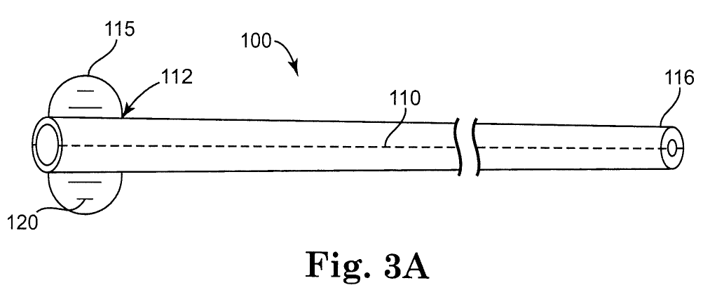

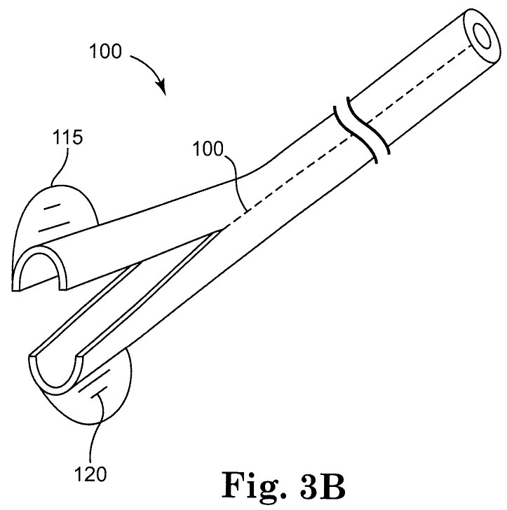

FIGS. 3A and 3B are perspective views of the outer sheath 60 according to various embodiments of the present invention. According to various embodiments of the present invention, the outer sheath 60 is a splittable sheath such that the sheath can be removed over the connector located at the proximal end of the lead where the connector has an outer diameter greater than the inner diameter of the sheath 60. The splittable outer sheath 60 is adapted to be removed from the outer surface 88 of the lead body 64 once the lead 54 has been secured at the implantation site. As shown in FIGS. 3A and 3B, the splittable outer sheath 60 includes at least one line of perforations longitudinally extending from a proximal end 112 of the sheath 60 to a distal end 116 of the sheath 100. The line of perforations 110 is adapted for splitting and removing the sheath 60 from the about outer surface 88 of the lead body 64. According to various embodiments of the present invention, the sheath 60 can include other means for splitting the sheath 60 including at least one line of weakness, at least one line of score marks, and/or at least one peel-away strip. In some embodiments, as shown in FIG. 3B, two or more lines of perforations are disposed 180 degrees from each other such that the sheath 60 can be removed in two or more sections. In the illustrated embodiment, the sheath 60 includes tabs or wings 115 located at the proximal end 112 of the sheath 60 to assist the physician with splitting the sheath 60 for removal of the sheath 60 from about the lead body. The tabs and/or wings 115 can include one or more grip marks 120 to facilitate gripping of the outer sheath 60 during removal from the outer surface 88 of the cardiac lead body 54.

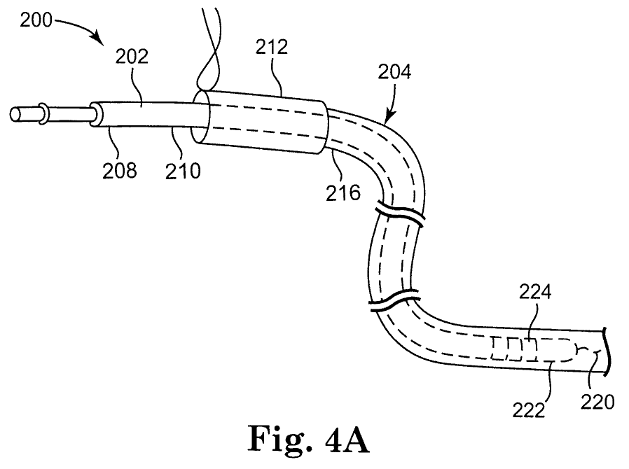

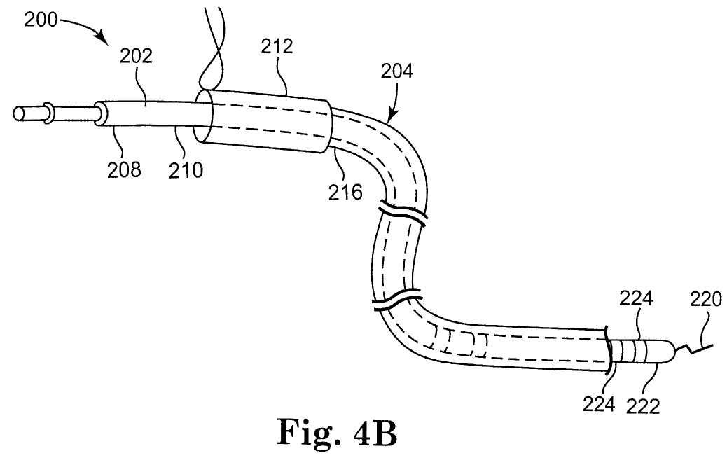

FIGS. 4A and 4B are schematic views of a cardiac lead assembly 200 according to other embodiments of the present invention. The cardiac lead assembly 200 includes a cardiac lead 202 and an outer sheath 204 disposed over a majority of the outer surface 208 of the cardiac lead body 210. As shown in FIGS. 4A and 4B, the outer sheath 204 includes a suture sleeve 212 located at a proximal end 216 of the outer sheath 204. Like the embodiments discussed above, the outer sheath 204 is slideably disposed over the outer surface 208 of the lead body, and is adapted to be movable by a physician to an extended position in which the outer sheath 204 is disposed over a fixation helix 220 located at the distal end 222 of the lead body 210, as shown in FIG. 4A, and a retracted position in which the outer sheath 204 has been retracted to expose the fixation helix 220 and/or any electrodes 224 located at the distal end 222 of the lead body 210, as shown in FIG. 4B. The suture sleeve 212 facilitates securing the outer sheath 200 in the retracted position upon chronic implantation of the cardiac lead 202 at the desired location within the patient's heart. According to some embodiments, as discussed above, the outer sheath 204 includes one or more means adapted for splitting the sheath, such as those described above in with respect to the sheath 60, which facilitate removal of the sheath 204 if necessary or desired.

According to various embodiments of the present invention, the outer sheath is configured such that the handling characteristics of the cardiac lead are comparable to those of a conventional cardiac lead. In some embodiments, the outer sheath has substantially the same flexibility and suppleness as the lead body itself.

According to some embodiments, the outer sheath can be fabricated from the same or similar material as the portion of the lead body over which it is disposed. The outer sheath can be fabricated from a variety of suitable biocompatible polymers or copolymers. According to further embodiments different polymeric materials may be used to fabricate different sections and/or regions of the outer sheath. In various embodiments, the polymer(s) used to fabricate the outer sheath should are selected such that the flexibility of the sheath is comparable to the flexibility of the cardiac lead body. Additionally, the selected polymer(s) should have sufficient tensile strength so as to resist tearing during implantation of the cardiac lead. Exemplary biocompatible polymers include polyurethane, polyetherketone (PEEK), polyether block amide (PBAX), PTFE, EFTFE, and polyimide. According to other embodiments, the outer sheath can include portions including an electrically transparent polymer such as ePTFE. This embodiment allows the outer sheath to remain in place over the electrodes while pacing thresholds are being evaluated prior to implantation of the lead.

According to various embodiments of the present invention, the wall thickness of the outer sheath is selected such that the presence of the outer sheath does not affect the handling characteristics of the cardiac lead over which the outer sheath is disposed. Additionally, the wall thickness of the outer sheath is such that it allows the outer sheath to be easily spit and removed from about the lead body via the one or more means adapted for splitting the outer sheath. According to some embodiments, the outer sheath has a wall thickness that does not substantially increase the overall diameter of the cardiac lead assembly from a first size to a second size. According to some embodiments, a wall thickness of the outer sheath ranges from about 0.005 inches to about 0.015 inches.

In each of the embodiments, discussed above, the cardiac lead assembly can include a lubricious coating. According to some embodiments, the lubricious coating can be disposed over an outer surface of the lead body. In other embodiments, an inner surface of the outer sheath can include the lubricious coating. The lubricious coating facilitates the retraction and/or removal of the outer sheath. Exemplary lubricious coatings include sil-glide and parylene.

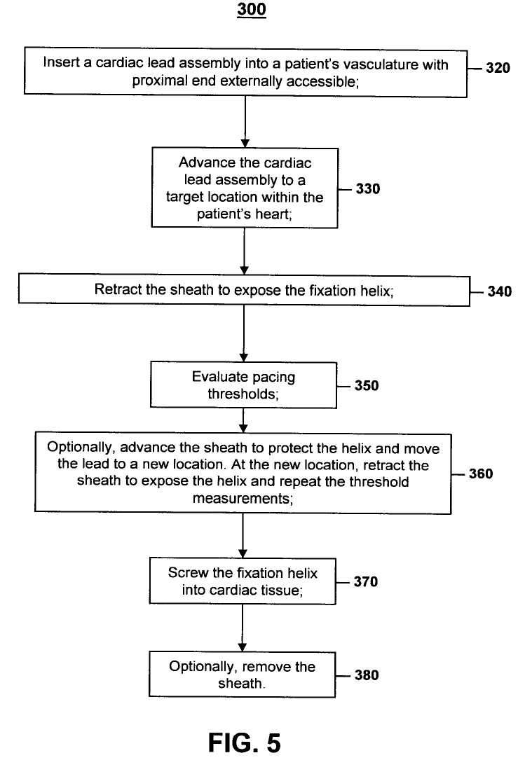

FIG. 5 is a flow chart of an exemplary method 300 of delivering the cardiac lead assembly to a target location within a patient's heart. The cardiac lead assembly is inserted into and advanced with a patient's vasculature such that the proximal end of the sheath is external to the patient's vasculature such that it is accessible to the physician or clinician performing the procedure (block 320 and block 330). Once the distal end of the lead assembly has reached the target location, the outer sheath can be retracted (block 340). Subsequently, pacing thresholds are then evaluated (block 350). According to some embodiments, if the pacing thresholds indicate that the position of the distal end of the lead needs to be adjusted, the sheath can be advanced over the fixation helix such that the helix is protected, the lead position adjusted, and the pacing thresholds re-evaluated (block 360). If the pacing thresholds are sufficient, the sheath is retracted and the fixation helix is screwed into the cardiac tissue at the target location (block 370). In some embodiments, the sheath is removed by splitting the sheath into two or more sections (block 380). According to other embodiments, the retracted sheath can then be secured in place using a suture sleeve for chronic implantation. The sheath can then be removed at a later time, if desired, by splitting and removing the sheath from about the cardiac lead. In some embodiments, a guide catheter coupled to an introducer may also be used to aid in the delivery of the cardiac lead assembly.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A cardiac rhythm management system comprising:
a pulse generator;
a cardiac lead assembly adapted to be coupled to the pulse generator, the assembly including:
a cardiac lead including:
an elongate lead body having an outer surface and a proximal end and a distal end;
at least one electrode located on the lead body;
at least one conductor operatively connected to the at least one electrode; and
a fixation helix located at the distal end of the body, the fixation helix including a distal end; and
a retractable sheath slideably disposed over at least a portion of the outer surface of the lead body and the fixation helix, the sheath including proximal and distal ends, wherein the sheath is dimensioned such that the distal end of the sheath is positionable at least flush with the distal end of the fixation helix.

2. The system according to claim 1, wherein the sheath is dimensioned to be disposed over about 50% to about 80% of the outer surface of the lead body.

3. The system according to claim 1, wherein the sheath has at least one line of weakness adapted to facilitate splitting of the sheath for removal of the sheath from the outer surface of the lead body.

4. The system according to claim 1, wherein the sheath has a pair of wings located at the proximal end of the sheath.

5. The system according to claim 1, wherein the sheath has a wall thickness ranging from 0.005 inches to about 0.015 inches.

6. The system according to claim 1, wherein the fixation helix is fixed relative to the distal end of the lead body.

7. The system of according to claim 1, wherein the outer surface of the lead body includes a lubricious coating to facilitate movement of the sheath relative to the lead body.

8. The system according to claim 1, wherein the sheath includes at least one means for splitting the sheath longitudingally extending from the proximal end to the distal end of the sheath.

9. The system according to claim 8 wherein the means for splitting the sheath includes any one of a line of weakness, a line of perforations, a peel-away strip, or a line of score marks.

10. The system according to claim 1, wherein the sheath includes an inner surface and a lubricious coating disposed over the inner surface of the sheath.

11. The system according to claim 1, wherein the sheath is adapted to be moved between at least a first position wherein the sheath is disposed over the fixation helix and at least a second position wherein the fixation helix is fully exposed.

12. The system according to claim 1, wherein the sheath includes a suture sleeve adapted to secure the sheath in a retracted position wherein the fixation helix is fully exposed.

13. The system according to claim 1, wherein the distal end of the sheath is dimensioned to extend beyond the distal end of the fixation helix when the sheath is in an extended position.

14. The system according to claim 1, wherein a distal end of the sheath is flush with a distal end of the fixation helix.

15. The system according to claim 1, wherein the sheath comprises at least one aperture adapted to be aligned with the at least one electrode.

16. The system according to claim 1, wherein the sheath has substantially similar flexibility as the lead body such that it does not substantially affect the handling characteristics of the lead body.

17. The system according to claim 1, wherein at least a portion of the sheath comprises an electrically transparent material.

18. A method of delivering a cardiac lead to a target location within a patient's heart comprising:
introducing a cardiac lead assembly into the patient's vasculature, the cardiac lead assembly including:
a cardiac lead including a lead body having a distal end, and a fixation helix located at the distal end of the body and including a helix distal end, and
a retractable sheath disposed over the lead body including a sheath proximal end and a sheath distal end, the sheath distal end positioned at least flush with the distal end of the fixation helix;
advancing the cardiac lead assembly to the target location within a patient's heart, while maintaining the proximal end of the sheath accessible from a location external to the patient's vasculature;
retracting the sheath to expose the fixation helix;
evaluating pacing thresholds at the target location; and
screwing the fixation helix into cardiac tissue at the target location.

19. The method according to claim 18, further comprising removing the retractable sheath.

20. The method according to claim 18, wherein introducing the cardiac lead assembly into the patient's vasculature includes inserting the cardiac lead assembly into the patient's vasculature via an introducer.

21. The method according to claim 18, wherein introducing the cardiac lead assembly into the patient's vasculature includes introducing the cardiac lead assembly into the patient's vasculature via a guide catheter.

22. The method according to claim 18, further comprising after evaluating pacing thresholds at the target implantation site, advancing the sheath over the fixation helix such that the sheath distal end is at least flush with the fixation helix distal end, repositioning the distal end of the lead body at a new target location, retracting the sheath to expose the fixation helix, and re-evaluating pacing thresholds at the new target location.

23. A cardiac lead assembly comprising:
a cardiac lead including an elongate lead body having an outer surface and a proximal end and a distal end;
at least one electrode located on the lead body;
at least one conductor operatively connected to the at least one electrode; and
a fixation helix located at the distal end of the body, the fixation helix including a distal end; and
a sheath including disposed over at least a portion of the outer surface of the lead body and the fixation helix, the sheath having proximal and distal ends and including splitting means for longitudinally splitting the sheath between the proximal and distal ends.
